(19) United States
(12) Patent Application Publication
(10) Pub. No.: US 2024/0053053 A1
(43) Pub. Date: Feb. 15, 2024

(54) Air Purifier

(71) Applicant: Brother Kogyo Kabushiki Kaisha, Nagoya (JP)

(72) Inventors: Naokatsu Osawa, Nagoya (JP); Ryuta Iijima, Nagoya (JP); Keisuke Omori, Nagoya (JP); Masashi Ichihashi, Mizuho (JP); Junji Yamano, Kariya (JP)

(21) Appl. No.: 18/495,410

(22) Filed: Oct. 26, 2023

Related U.S. Application Data
(63) Continuation of application No. PCT/JP2022/013018, filed on Mar. 22, 2022.

(30) Foreign Application Priority Data
Apr. 28, 2021 (JP) 2021-076653

(51) Int. Cl. F24F 13/28; F24F 8/10; F24F 13/08; F24F 13/20

(52) U.S. Cl. CPC F24F 13/28; F24F 8/10; F24F 13/08; F24F 13/20; F24F 2013/205

(57) ABSTRACT

An air purifier includes a housing having an air inlet, an air outlet, and a cavity portion connecting the air inlet and the air outlet, two blowers disposed in the cavity portion and each including a fan rotatable to generate a flow of air, and a filter disposed in the cavity portion. During rotation of the fan, the two blowers each suck in air from outside of the housing through the air inlet into the cavity portion, and blow the air having passed through the filter from the air outlet to the outside of the housing. A direction in which the air inlet is open crosses a direction in which the air outlet is open. An opening area of the air outlet is smaller than an opening area of the air inlet.

### REFERENCE TO RELATED APPLICATIONS

This is a continuation application of International Application No. PCT/JP2022/013018 filed on Mar. 22, 2022, which claims priority from Japanese Patent Application No. 2021-076653 filed on Apr. 28, 2021. The entire contents of the aforementioned applications are incorporated herein by reference.

### BACKGROUND ART

A known air purifier includes a main body having an air inlet and an air outlet, and a blower housed in the main body. The blower sucks in air outside of the main body through the air inlet and blows the air from the air outlet to the outside of the main body.

### DESCRIPTION

In the above-described air purifier, air blown from the air outlet may be sucked in again from the air inlet. Since the air blown out from the air outlet is purified air, there is a possibility that the suction efficiency of the unpurified air is reduced in the air purifier.

Aspects of the disclosure provide an air purifier capable of reducing the likelihood that air blown from an air outlet is sucked in again from an air inlet.

According to an aspect of the disclosure, an air purifier includes a housing having an air inlet, an air outlet, and a cavity portion connecting the air inlet and the air outlet, two blowers disposed in the cavity portion and each including a fan rotatable to generate a flow of air, and a filter disposed in the cavity portion. During rotation of the fan, the two blowers each suck in air from outside of the housing through the air inlet into the cavity portion, and blow the air having passed through the filter from the air outlet to the outside of the housing. A direction in which the air inlet is open crosses a direction in which the air outlet is open. An opening area of the air outlet is smaller than an opening area of the air inlet.

Further, since the opening area of the air outlet is smaller than the opening area of the air inlet, the air velocity is higher at the air outlet than at the air inlet. Therefore, the air blown from the air outlet is further away from the air inlet as compared with a case where the air velocity is lower at the air outlet than at the air inlet. The air purifier can thus reduce the likelihood that the air blown out from the air outlet is sucked in again from the air inlet.

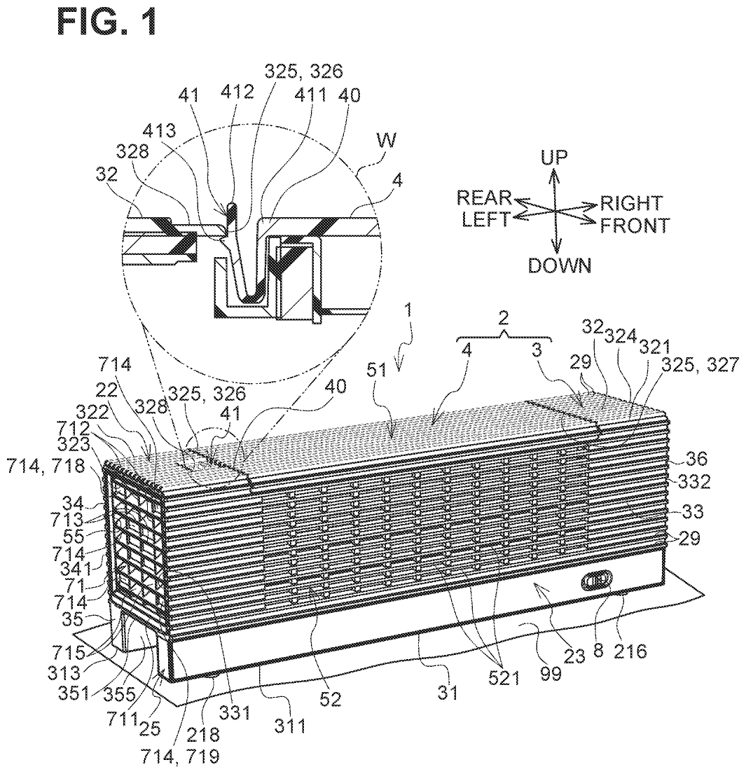

FIG. 1 is a perspective view of an air purifier as viewed from above.

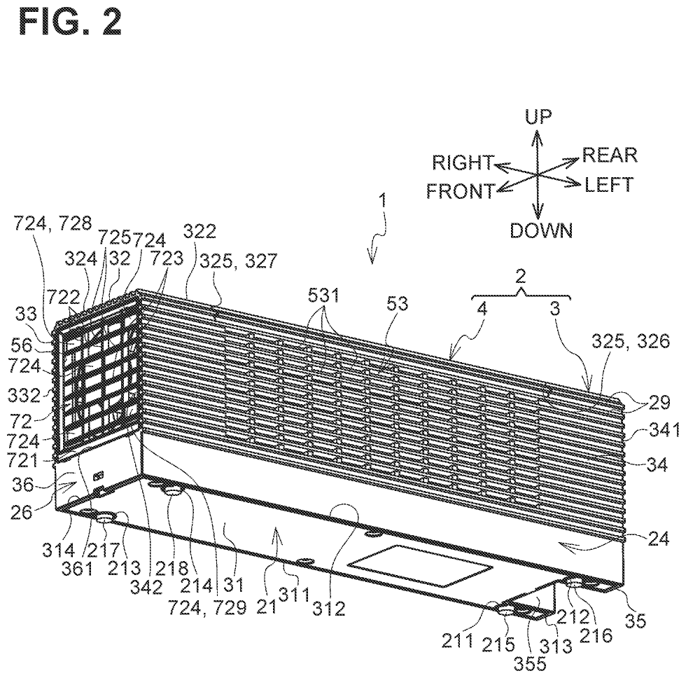

FIG. 2 is a perspective view of an air purifier as viewed from above.

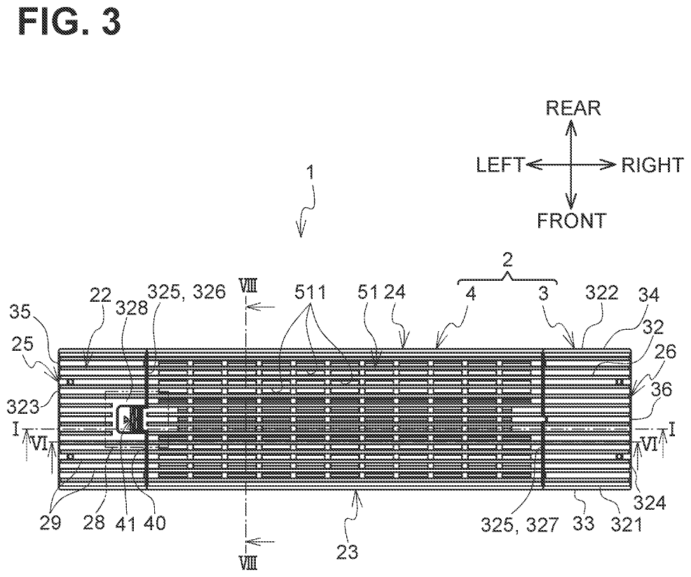

FIG. 3 is a plan view of the air purifier.

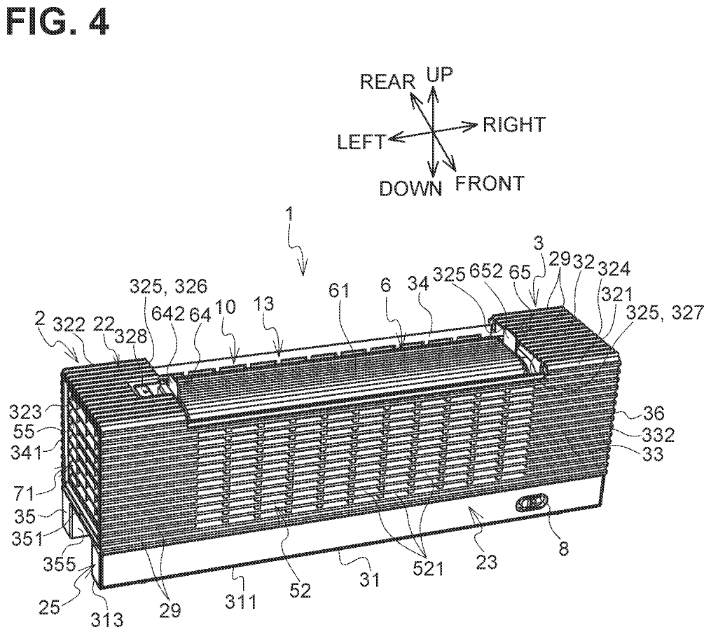

FIG. 4 is a perspective view of the air purifier without a lid illustrated in FIG. 1, as viewed from above.

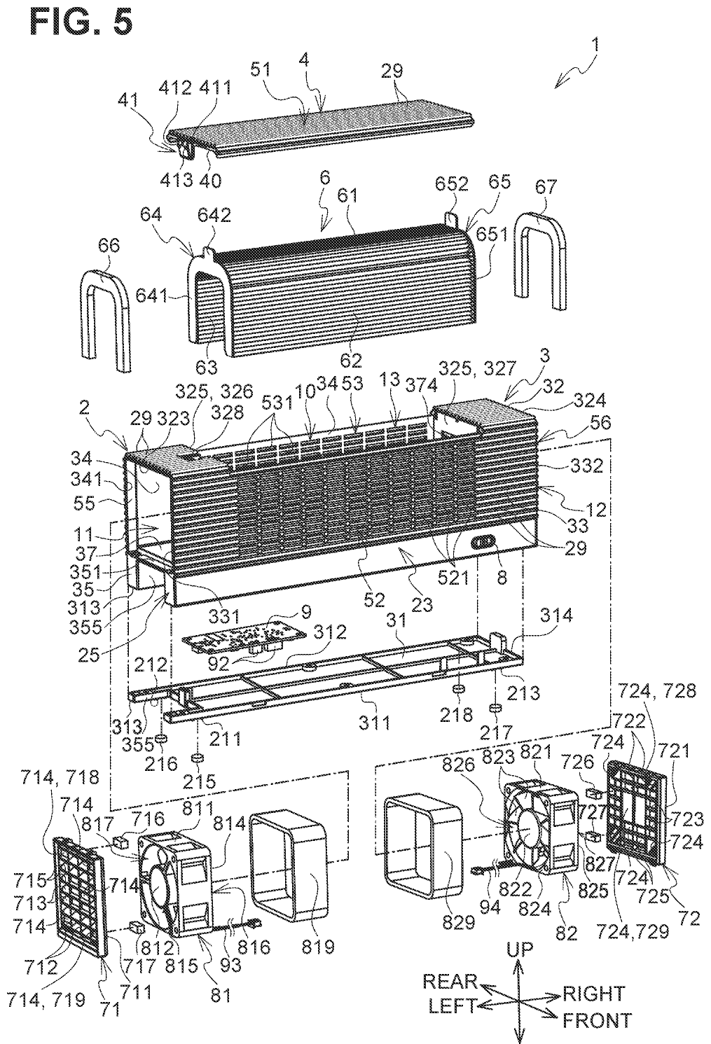

FIG. 5 is an exploded perspective view of the air purifier as viewed from above.

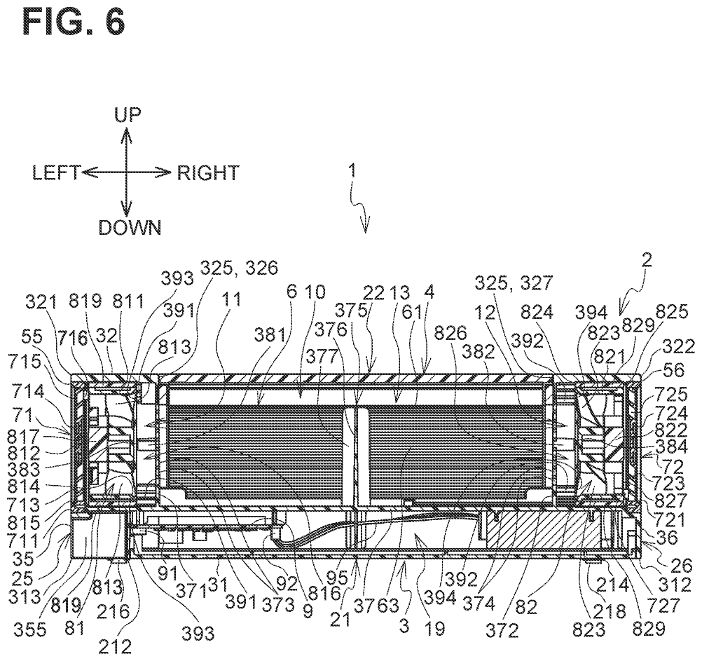

FIG. 6 is a cross-sectional view taken along line VI-VI of FIG. 3.

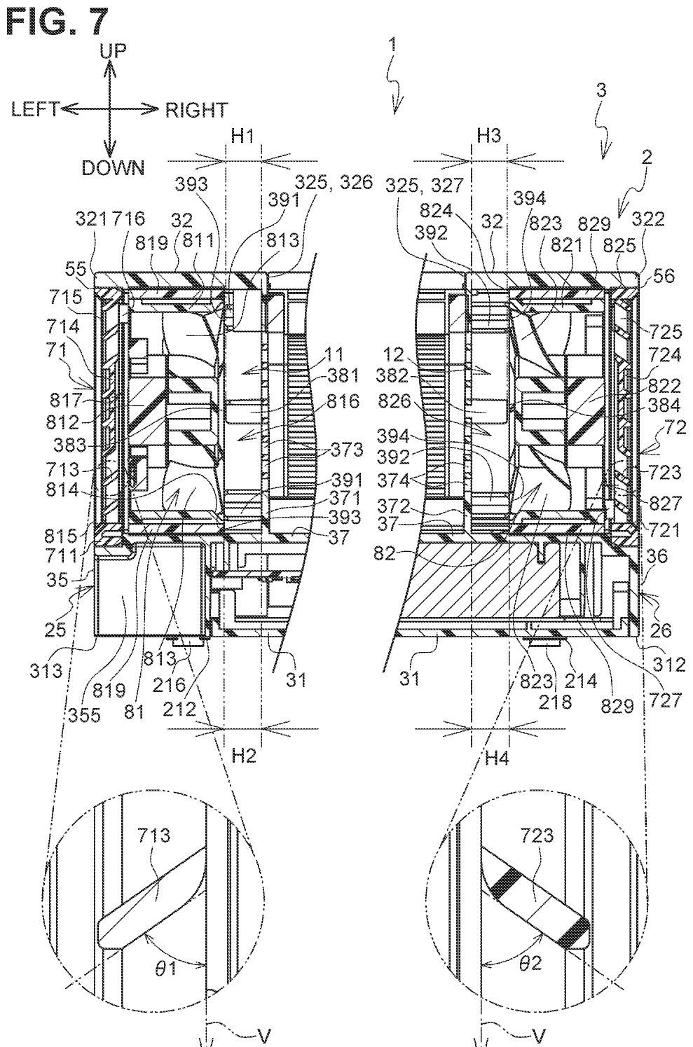

FIG. 7 is a partially enlarged cross-sectional view taken along line VII-VII illustrated in FIG. 3, illustrating left and right end portions of the air purifier.

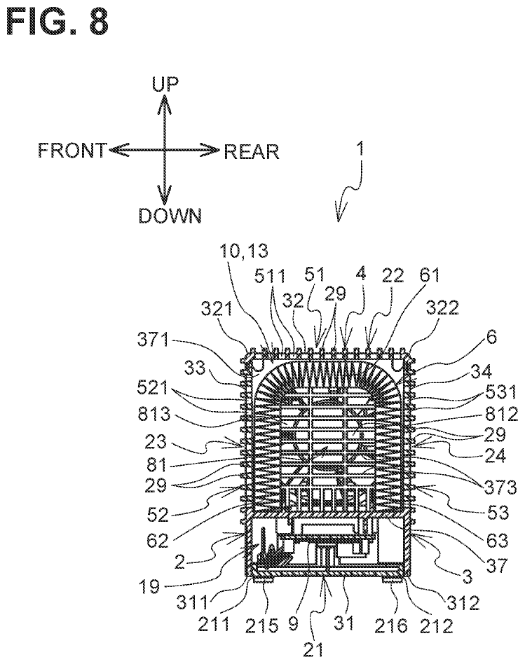

FIG. 8 is a cross-sectional view taken along line VIII-VIII of FIG. 3.

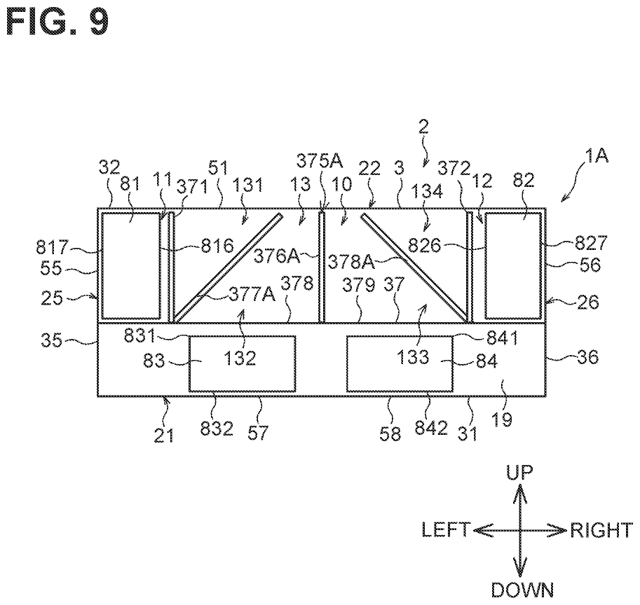

FIG. 9 is a schematic view of an air purifier as viewed from front.

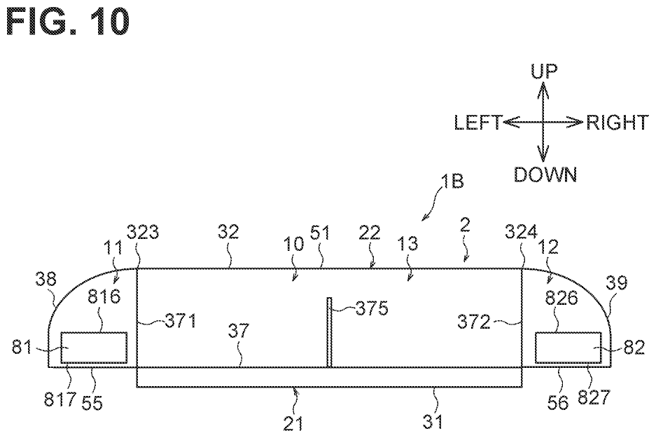

FIG. 10 is a schematic view of an air purifier as viewed from front.

An air purifier 1 according to an embodiment of the present disclosure will be described with reference to the drawings. In the following description, the expressions “up”, “down”, “front”, “rear”, “right”, and “left” in FIG. 1 are used to define the various parts when an air purifier 1 is disposed in an orientation in which it is intended to be used.

Housing 2

As illustrated in FIGS. 1 to 3, the air purifier 1 includes a housing 2. The housing 2 has a rectangular parallelepiped shape, is longer in the up-down direction than in the front-rear direction, and is longer in the left-right direction than in the up-down direction. The housing 2 includes a case 3 and a lid 4. The case 3 includes a lower wall 31, an upper wall 32, a front wall 33, a rear wall 34, a left wall 35, and a right wall 36. The lower wall 31, the upper wall 32, the front wall 33, the rear wall 34, the left wall 35, and the right wall 36 are plates and define a lower surface 21, an upper surface 22, a front surface 23, a rear surface 24, a left surface 25, and a right surface 26 of the housing 2, respectively.

The lower wall 31 illustrated in FIG. 2 and the upper wall 32 illustrated in FIG. 1 extend in the front-rear and right-left directions and face each other in the up-down direction. As illustrated in FIG. 1, the front wall 33 extends from a front end 311 of the lower wall 31 to a front end 321 of the upper wall 32. As illustrated in FIG. 2, the rear wall 34 extends from a rear end 312 of the lower wall 31 to a rear end 322 of the upper wall 32. The front wall 33 and the rear wall 34 face each other in the front-rear direction.

As illustrated in FIG. 1, the left wall 35 extends from a left end 313 of the lower wall 31 to a position below a left end 323 of the upper wall 32. In the present embodiment, an upper end 351 of the left wall 35 is located at a position of about ¼ of the height from the lower wall 31 to the upper wall 32 in the up-down direction. The left wall 35 has a recess 355. The recess 355 is recessed rightward from the left wall 35.

As illustrated in FIG. 2, the left wall 36 extends from a right end 314 of the lower wall 31 to a position below a right end 324 of the upper wall 32. In the present embodiment, an upper end 361 of the right wall 36 is located in a lower one fourth of a height from the lower wall 31 to the upper wall 32 in the up-down direction, and is located at the same height as the upper end 351 of the left wall 35 illustrated in FIG. 1. The left wall 35 and the right wall 36 face each other in the left-right direction.

As illustrated in FIG. 4, the upper wall 32 has an opening 325. The opening 325 has a rectangular shape in plan view, and is longer in the left-right direction than in the front-rear direction. The opening 325 extends from the front end 321 of the upper wall 32 to the rear end 322 of the upper wall 32, and extends from a position further to the right than the left end 323 of the upper wall 32 to a position further to the left than the right end 324 of the upper wall 32.

As illustrated in FIG. 1, the lid 4 is a plate and has a shape corresponding to the shape of the opening 325 in a plan view. In the embodiment, the lid 4 has a rectangular shape and is longer in the left-right direction than in the front-rear direction. The lid 4 is detachable from the case 3. The lid 4 attached to the case 3 closes the opening 325. In this state, the lid 4 forms an upper surface 22 of the housing 2 together with the upper wall 32. As illustrated in FIG. 4, the opening 325 is exposed upward in a state where the lid 4 illustrated in FIG. 1 is removed from the case 3. In the following description, unless otherwise specified, a state where the lid 4 is attached to the case 3 (refer to FIGS. 1 to 3) will be described as a reference.

As illustrated in FIG. 1, the lid 4 includes a grip portion 41 located at a left end 40 of the lid 4. An enlarged view W illustrates an end view of the grip portion 41 and the periphery of the grip portion 41 in the direction of the arrow I-I illustrated in FIG. 3. When viewed in cross section taken along a plane orthogonal to the front-rear direction, the grip portion 41 has a U-shape opening upward and extends from a first end 411 to a second end 412. The first end 411 is connected to a central portion of the left end 40 of the lid 4 in the front-rear direction. The second end 412 is located above the first end 411 and a planar area 328 to be described later.

The grip portion 41 includes a locking piece 413 below the second end 412. The locking piece 413 protrudes leftward from a position below the second end 412 in the grip portion 41. The grip portion 41 is elastically deformable in the left-right direction so that the second end 412 moves to or away from the first end 411. The grip portion 41 is a snap fit to maintain a state where the lid 4 is attached to the case 3 when the locking piece 413 is in engagement with a bottom of the upper wall 32.

As illustrated in FIGS. 1 to 3, the housing 2 has an upper surface 22, a front surface 23, and a rear surface 24 each provided with a plurality of ribs 29. The ribs 29 extend in the left-right direction. The ribs 29 on the upper surface 22 of the housing 2 protrude upward and are arranged in the front-rear direction. The ribs 29 on the front surface 23 of the housing 2 protrude frontward and are arranged in the vertical direction. The ribs 29 on the rear surface 24 of the housing 2 protrude rearward and are arranged in the front-rear direction.

As illustrated in FIG. 3, the housing 2 has an affixture portion 28. The affixture portion 28 is a predetermined area astride the boundary between the upper wall 32 and the lid 4. The affixture portion 28 includes the planar area 328 of the upper surface 22 of the housing 2. The planar area 328 is located to the left of a left end 326 of the opening 325 at the center of the upper surface 22 of the housing 2 in the front-rear direction, and has a rectangular shape in a plan view. The planar area 328 is located at a portion of the upper wall 32 to overlap the locking piece 413 for engaging in a plan view (refer to FIG. 1). The planar area 328 has no ribs 29. The planar area 328 is thus superior to any other area on the upper surface 22 of the housing 2 where the ribs 29 are provided, in terms of adhesion of a label (not illustrated).

An air inlet 51 is defined in the upper surface 22 of the housing 2. Specifically, the air inlet 51 is defined in the lid 4, and has a plurality of openings 511. The openings 511 are arranged in a grid pattern in the front-rear and left-right directions. The openings 511 extend through the lid 4 in the up-down direction. Thus, a direction in which the air inlet 51 is open is the up-down direction.

As illustrated in FIG. 1, an air inlet 52 is defined in the front surface 23 of the housing 2. The air inlet 52 is located between the left end 326 and the right end 327 of the opening 325 in the left-right direction, and is constituted by a plurality of openings 521. The openings 521 are arranged in a grid pattern in the up-down and left-right directions and extend through the front wall 33 in the front-rear direction. Thus, a direction in which the air inlet 52 is open is the front-rear direction and is orthogonal to the direction (up-down direction) in which the air inlet 51 is open.

As illustrated in FIG. 2, an air inlet 53 is defined in the rear surface 24 of the housing 2. The air inlet 53 is located between the left end 326 and the right end 327 of the opening 325 in the left-right direction, and has a plurality of openings 531. The openings 531 are arranged in a grid pattern in the up-down and left-right directions and extend through the front wall 34 in the front-rear direction. Thus, a direction in which the air inlet 53 is open is the front-rear direction and is orthogonal to the direction (front-rear direction) in which the air inlet 52 is open. That is, the direction (front-rear direction) in which the air inlet 53 is open is orthogonal to the direction (up-down direction) in which the air inlet 51 is open. Air outside the housing 2 is drawn in the housing 2 through the air inlets 51, 52, and 53 by driving of blowers 81 and 82 (refer to FIG. 5) to be described later.

As illustrated in FIG. 1, an air outlet 55 is defined in the rear surface 25 of the housing 2. The air outlet 55 is an opening and has a rectangular shape in a left side view. The air outlet 55 is defined by the left end 323 of the upper wall 32, a left end 331 of the front wall 33, a left end 341 of the rear wall 34, and the upper end 351 of the left wall 35. Thus, a direction in which the air outlet 55 is open is the left-right direction, and is orthogonal to the direction (the up-down direction or the front-rear direction) of each of the air inlets 51, 52, and 53. Air inside the housing 2 is blown out from the air outlet 55 to the outside of the housing 2 by the driving of the blower 81 (refer to FIG. 5) described later.

As illustrated in FIG. 2, an air outlet 56 is defined in the right surface 26 of the housing 2. The air outlet 56 is an opening and has a rectangular shape in a right side view. The air outlet 56 is defined by the right end 324 of the upper wall 32, a right end 332 of the front wall 33, a right end 342 of the rear wall 34, and the upper end 361 of the left wall 36. Thus, a direction in which the air outlet 56 is open is the left-right direction, and is parallel to the direction (left-right direction) in which the air outlet 55 is open. That is, the direction (left-right direction) in which the air outlet 56 is open is orthogonal to the direction (the up-down direction or the front-rear direction) in which each of the air inlets 51, 52, and 53 is open. Air inside the housing 2 is blown out from the air outlet 56 to the outside of the housing 2 by driving of the blower 82 (refer to FIG. 5) described later.

In the embodiment, an opening area of the air outlet 55 illustrated in FIG. 1 is smaller than an opening area of the air inlets 51, 52, and 53 illustrated in FIGS. 1 to 3. In the embodiment, an opening area of the air outlet 56 illustrated in FIG. 2 is smaller than the opening area of the air inlets 51, 52, and 53 illustrated in FIGS. 1 to 3.

The opening area of the air outlet 55 illustrated in FIG. 1 is an area of a space surrounded by the left end 323 of the upper wall 32, the left end 331 of the front wall 33, the left end 341 of the rear wall 34, and the upper end 351 of the left wall 35. The opening area of the air outlet 56 illustrated in FIG. 2 is an area of a space surrounded by the right end 324 of the upper wall 32, the right end 332 of the front wall 33, the right end 342 of the rear wall 34, and the upper end 361 of the left wall 36. The opening area of the air inlets 51, 52, and 53 illustrated in FIGS. 1 to 3 is the sum of the areas of the openings 511 illustrated in FIG. 3, the openings 521 illustrated in FIG. 1, and the openings 531 illustrated in FIG. 2.

As illustrated in FIG. 2, the lower surface 21 of the housing 2 has recesses 211, 212, 213, and 214. The recesses 211, 212, 213, and 214 are each located at one of the four corners of the lower surface 21 and are recessed upward from the lower surface 21. The recesses 211 and 213 are located at the same relative positions in the front-rear direction. The recesses 212 and 214 are located at the same relative positions in the front-rear direction. The recesses 211 and 212 are located at the same relative positions in the left-right direction. The recesses 213 and 214 are located at the same relative positions in the left-right direction. Specifically, the recesses 211 and 212 are located at positions overlapping with a blower 81 described later in a plan view (refer to FIG. 6). The recesses 213 and 214 are located at positions overlapping with a blower 82 described later in a plan view (refer to FIG. 6).

As illustrated in FIGS. 2 and 5, second elastic members 215, 216, 217, and 218 are bonded to the recesses 211, 212, 213, and 214, respectively. The second elastic members 215 and 216 are thus located directly below the air blower 81 described later (refer to FIG. 6). The second elastic members 217 and 218 are located directly below the blower 82 described later (refer to FIG. 6).

The second elastic members 215, 216, 217, and 218 each have a columnar shape and protrude further downward than the lower surface 21 of the housing 2. In the present embodiment, the second elastic members 215, 216, 217, and 218 are made of rubber, and are made of urethane foam having a 25 percent compression-deflection value of 0.133 N/mm2 or less per unit area. The 25% compression-deflection value is a measure of pressure required to compress a member by 25% of its original thickness.

In the embodiment, a coefficient of static friction of each of the second elastic members 215, 216, 217, and 218 is greater than a coefficient of static friction of the lower surface 21 of the housing 2. The coefficient of static friction of each of the second elastic members 215, 216, 217, and 218 is greater than a coefficient of static friction of the bottom surface of the recess 211. The coefficient of static friction of each of the second elastic members 215, 216, 217, and 218 is of such a magnitude that the housing 2 does not move relative to an installation surface 99 even if the user does not support the housing 2 when a slide switch 8 described later is operated by the user.

As illustrated in FIGS. 5 to 8, the case 3 includes a middle wall 37. The middle wall 37 is located between the lower wall 31 and the upper wall 32 in the up-down direction. The middle wall 37 is a plate and extends from the upper end 351 of the left wall 35 to the upper end 361 of the right wall 36. The middle wall 37 faces each of the lower wall 31 and the upper wall 32 in the up-down direction.

As illustrated in FIG. 6, a cavity portion 10 and an accommodating portion 19 are defined inside the housing 2. The cavity portion 10 is a space surrounded by the upper wall 32, the front wall 33, the rear wall 34, and the middle wall 37. As illustrated in FIG. 4, the upper end of the cavity portion 10 is connected to the air inlet 51 illustrated in FIG. 1. The front end of the cavity portion 10 is connected to the air inlet 52. The rear end of the cavity portion 10 is connected to the air inlet 53 illustrated in FIG. 2. As illustrated in FIG. 6, the left end of the cavity portion 10 is connected to the air outlet 55. The right end of the cavity portion 10 is connected to the air outlet 56. The accommodating portion 19 is a space surrounded by the lower wall 31, the front wall 33 illustrated in FIG. 1, the rear wall 34 illustrated in FIG. 2, the left wall 35, the right wall 36, and the middle wall 37.

As illustrated in FIGS. 6 and 7, the middle wall 37 is provided with partition walls 371 and 372 and a partition portion 375. The partition wall 371 is located at the left end 326 of the opening 325 in the left-right direction. The partition wall 372 is located at the right end 327 of the opening 325 in the left-right direction. The partition walls 371 and 372 are plates and extend from the front wall 33 to the rear wall 34 and from the middle wall 37 to the upper wall 32 illustrated in FIG. 5.

The partition wall 371 has a plurality of slits 373 (refer to FIG. 8). The slits 373 extend through the partition wall 371 in the left-right direction. The partition wall 372 has a plurality of slits 374 (refer to FIG. 5). The slits 374 extend through the partition wall 371 in the left-right direction.

The partition walls 371 and 372 face each other in the left-right direction and partition the cavity portion 10 into a left cavity portion 11, a right cavity portion 12, and a middle cavity portion 13. The left cavity portion 11 is a space to the left of the partition wall 371 in the cavity portion 10. The right cavity portion 12 is a space to the right of the partition wall 372 in the cavity portion 10. The middle cavity portion 13 is a space between the partition walls 371 and 372 in the left-right direction in the cavity portion 10, and is located below the lid 4. The left cavity portion 11 and the middle cavity portion 13 are connected to each other via the slits 373. The right cavity portion 12 and the middle cavity portion 13 are connected to each other via the slits 374.

As illustrated in FIG. 7, the partition wall 371 includes a protrusion 381 and a plurality of contact portions 391 of equal length. The protrusion 381 protrudes leftward from a central portion of the partition wall 371 in a left side view. In the embodiment, four contact portions 391 each protrude leftward from a corner of four corners of the partition wall 371. FIG. 6 illustrates the rear two of the four contact portions 391. In the left-right direction, a length H1 from the partition wall 371 to a distal end 383 of the protrusion 381 is shorter than a length H2 from the partition wall 371 to a distal end 393 of each contact portion 391.

The partition wall 372 is provided with a protrusion 382 and a plurality of contact portions 392 of equal length. The protrusion 382 protrudes rightward from a central portion of the partition wall 372 in a left side view. In the embodiment, four contact portions 392 each protrude rightward from a corner of four corners of the partition wall 372. FIGS. 6 and 7 illustrate the rear two of the four contact portions 392. In the left-right direction, a length H3 from the partition wall 372 to a distal end 384 of the protrusion 382 is shorter than a length H4 from the partition wall 372 to a distal end 394 of each contact portion 392.

As illustrated in FIG. 6, the partition portion 375 is located in the middle cavity portion 13 and has partition walls 376 and 377. The partition walls 376 and 377 have a plate shape and extend upward from the middle wall 37 to a position below the lid 4. The partition walls 376 and 377 have equal vertical lengths.

The partition wall 376 is located in the center of the middle cavity portion 13 in the left-right direction. The partition wall 376 extends in the front-rear direction between a position to the rear of the front wall 33 and a position to the front of the rear wall 34. In the front-rear direction, the partition wall 376 has a length greater than or equal to half of a distance between the front wall 33 and the rear wall 34, and specifically, the length is ½ to ⅗ of the distance between the front wall 33 and the rear wall 34. In the front-rear direction, a distance between the front end of the partition wall 376 and the front wall 33 and a distance between the rear end of the partition wall 376 and the rear wall 34 are respectively ⅕ to ¼ of the distance between the front wall 33 and the rear wall 34, and are shorter than the length of the partition wall 376. The partition wall 376 partitions the middle cavity portion 13 in the left-right direction.

The partition wall 377 is located in the center of the middle cavity portion 13 in the left-right direction. The partition wall 377 extends in the left-right direction between a position to the right of the partition wall 371 and a position to the left of the partition wall 372. In the embodiment, the length of the partition wall 377 in the left-right direction is approximately ⅓ of the length of the partition wall 376 in the front-rear direction. The partition wall 377 partitions the middle cavity portion 13 in the front-rear direction.

Blowers 81 and 82

As illustrated in FIGS. 5 and 7, the air purifier 1 includes two blowers 81 and 82. The blower 81 is disposed in the left cavity portion 11. The blower 81 is of an axial flow type and includes a support portion 811, a motor 812, and a fan 813 illustrated in FIG. 7. The support portion 811 is a casing and has a rectangular tubular shape in a left side view. Hereinafter, an area surrounded by a right end 814 of the support portion 811 is referred to as an intake portion 816, and an area surrounded by a left end 815 of the support portion 811 is referred to as an exhaust portion 817.

The motor 812 is fixed to the support portion 811 so as to be disposed in the center of the support portion 811 in a left side view. An output shaft of the motor 812 extends to the right from the motor 812. The fan 813 is disposed in the support portion 811 and is fixed to the output shaft of the motor 812. Thus, the rotation axis of the fan 813 extends in the left-right direction.

The axial direction (left-right direction) of the fan 813 is orthogonal to the direction (up-down direction) in which the air inlet 51 is open, the direction (front-rear direction) in which the air inlet 52 is open, and the direction (front-rear direction) in which the air inlet 53 is open. The axial direction (left-right direction) of the fan 813 is parallel to the direction (left-right direction) in which the air outlet 55 is open and the direction (left-right direction) in which the air outlet 56 is open.

The fan 813 rotates to generate a flow of air from the intake portion 816 toward the exhaust portion 817. Thus, the air flows from the right side to the left side of the blower 81. That is, the intake portion 816 is an area upstream of the fan 813 in the flow of air generated by the rotation of the fan 813, and the exhaust portion 817 is an area downstream of the fan 813 in the flow of air generated by the rotation of the fan 813.

The blower 81 is fixed to the case 3 via a seal member 819 in a state where the intake portion 816 is located to the right with respect to the exhaust portion 817. In this case, the distal ends 393 of the contact portions 391 are in contact with the right end 814 of the support portion 811. The distal ends 383 of the protrusions 381 are each located at a position spaced from the fan 813 to the right. The seal member 819 is made of soft urethane foam. The seal member 819 fills a gap between the peripheral wall of the support portion 811 and each of the upper wall 32, the front wall 33, the rear wall 34, and the middle wall 37. The seal member 819 improves the air suction efficiency of the blower 81.

The blower 82 is disposed in the right cavity portion 12 and has the same structure as the blower 81. That is, the blower 82 includes a support portion 821, a motor 822, and a fan 823. The support portion 821 corresponds to the support portion 811. The motor 822 corresponds to the motor 812. An output shaft of the motor 822 extends to the left from the motor 822. The fan 823 corresponds to the fan 813. The intake portion 826 is an area surrounded by a left end 824 of the support portion 821. The exhaust portion 827 is an area surrounded by a right end 825 of the support portion 821.

The seal member 829 corresponds to the seal member 819. The blower 82 is fixed to the case 3 via the seal member 829 in a state where the intake portion 826 is located to the right with respect to the exhaust portion 827. In this case, the distal ends 394 of the contact portions 392 are in contact with the left end 824 of the support portion 821. The distal ends 384 of the protrusions 382 are each located at a position spaced from the fan 823 to the left.

The axial direction (left-right direction) of the fan 823 is orthogonal to the direction (up-down direction) in which the air inlet 51 is open, the direction (front-rear direction) in which the air inlet 52 is open, and the direction (front-rear direction) in which the air inlet 53 is open. The axial direction (left-right direction) of the fan 823 is parallel to the direction (left-right direction) in which the air outlet 55 is open and the direction (left-right direction) in which the air outlet 56 is open, and is parallel to the axial direction (left-right direction) of the fan 813.

The fan 823 rotates to generate an air flow from the intake portion 826 toward the exhaust portion 827. Thus, the air flows from the left side to the right side of the blower 82. That is, the blowers 81 and 82 generate the flows of air in opposite directions.

In the embodiment, the two blowers 81 and 82 face each other in the left-right direction. In the embodiment, “the two blowers 81 and 82 face each other” means that the intake portions 816 and 826 face each other, that is, a state in which the two blowers 81 and 82 are disposed such that a direction from the intake portion 816 to the exhaust portion 817 (direction from right to left) and a direction from the intake portion 826 to the exhaust portion 827 (direction from left to right) are opposite to each other.

Louvers 71 and 72

As illustrated in FIGS. 5 and 7, the air purifier 1 includes louvers 71 and 72. The louver 71 is engaged in the air outlet 55, and includes a frame 711, a plurality of vertical plates 712, a plurality of slats 713, and a plurality of closing plates 714. The frame 711 has the same shape as the shape of the air outlet 55 in a left side view, and has a rectangular shape in the embodiment.

The vertical plates 712 extend in the up-down direction and are arranged in the front-rear direction in the frame 711. The slats 713 extend in the left-right direction and are arranged in the up-down direction in the frame 711. The slats 713 slope downward from right to left. As illustrated in FIG. 7, a reference line V extending from the upper side to the lower side in the direction in which the air inlet 51 is open is defined, and an angle coinciding with the reference line V is 0 degrees. In the embodiment, an angle θ1 of the slats 713 to the left relative to the reference line V is 55 degrees.

As illustrated in FIG. 5, the louver 71 has a plurality of openings 715 defined by the vertical plates 712 and the slats 713 in the frame 711. The closing plates 714 are provided at the openings 715 at the four corners and the opening 715 at the center among the plurality of openings 715. The user thus cannot visually recognize the inside of the housing 2 through the closing plates 714 in the left surface 25 of the housing 2 in the left side view. Thus, the design of the air purifier 1 is improved.

The air purifier 1 can prevent a foreign substance such as a stick or dust from entering the inside of the housing 2 at the closing plates 714 in the left surface 25 of the housing 2. The closing plates 714 blocking the openings 715 at the four corners and the center are less likely to affect the air velocity at the air outlet 55 than those blocking the other openings 715. Therefore, the closing plate 714 can prevent not only a decrease in the air velocity at the air outlet 55 but also the entry of a foreign substance such as a stick and dust into the housing 2 from the air outlet 55.

In the present embodiment, the opening area of the louver 71 is smaller than the opening area of the air inlets 51, 52, and 53. The opening area of the louver 71 is the sum of the areas of openings 715 that are not blocked by the closing plates 714 among the openings 715.

According to the above-described structure of the louver 71, when the air flows from the right side to the left side of the louver 71 via the openings 715, the air flows in a direction inclined leftward by 55 degrees relative to the reference line V by the slats 713 on the left side of the louver 71. In this way, the louver 71 guides the air, which is blown out from the air outlet 55 by the blower 81, downward to the outside of the housing 2.

The louver 71 is provided with first elastic members 716 and 717. The first elastic members 716 and 717 have a prismatic shape and are fixed to the right surface of the louver 71. Specifically, the first elastic member 716 is bonded to the right surface of the closing plate 718 at the upper rear corner among the closing plates 714. Specifically, the first elastic member 717 is bonded to the right surface of the closing plate 719 at the lower front corner among the closing plates 714. The first elastic members 716, 717 are sponges. The louver 71 and the blower 81 are in contact with the first elastic members 716 and 717 (refer to FIG. 7 for the first elastic member 716). In other words, the first elastic members 716 and 717 are located between and in contact with the louver 71 and the support portion 811.

The blower 81 is pressed from left to right by the elastic force of the first elastic members 716 and 717. The contact portions 391 receive the elastic force of the first elastic members 716 and 717 via the blower 81. Thus, the position of the blower 81 in the left-right direction is maintained.

The louver 72 is engaged in the air outlet 56 and has the same structure as the louver 71. That is, the louver 72 includes a frame 721, a plurality of vertical plates 722, a plurality of slats 723, and a plurality of closing plates 724. The frame 721 corresponds to the frame 711. The vertical plates 722 correspond to the vertical plates 712. The slats 723 correspond to the slats 713. An angle θ2 of the slats 723 to the right relative to the reference line V is 55 degrees. The closing plates 724 correspond to the closing plates 714. In this embodiment, the slats 723 are inclined to the right by 55 degrees relative to the reference line V.

The louver 72 has a plurality of openings 725. The openings 725 correspond to the openings 715. An opening area of the louver 72 is smaller than the opening area of the air inlets 51, 52, and 53. The opening area of the louver 72 is the sum of the areas of openings 725 that are not blocked by the closing plates 724 among the openings 725.

The louver 72 is provided with first elastic members 726 and 727. The first elastic members 726 and 727 correspond to the first elastic members 716 and 717. The first elastic member 726 is bonded to the left surface of the closing plate 728 at the upper front corner among the closing plates 724. The first elastic member 727 is bonded to the left surface of the closing plate 729 at the lower rear corner among the closing plates 724. The first elastic members 726 and 727 are located between and in contact with the louver 72 is in contact with the support portion 821 (refer to FIG. 7 for the first elastic member 727). The position of the blower 81 in the left-right direction is maintained by the elastic force of the first elastic members 716 and 717 and the contact portions 392.

Filter 6

As illustrated in FIGS. 4 to 8, the air purifier 1 includes a filter 6. The filter 6 is disposed in the middle cavity portion 13 and is detachable from the middle cavity portion 13. The filter 6 is a HEPA filter, which removes viruses, dirt, dust and other foreign substances from the air to clean the air. The filter 6 has a pleated structure and includes an upper filter 61, a front filter 62, and a rear filter 63.

As illustrated in FIG. 5, the upper filter 61 extends in the front-rear and left-right directions. The front filter 62 extends in the left-right direction and extends downward from the front end of the upper filter 61. The rear filter 63 extends in the left-right direction and extends downward from the rear end of the upper filter 61. The rear filter 63 faces the front filter 62 in the front-rear direction. Thus, when viewed in cross section taken along a plane orthogonal to the left-right direction, the filter 6 has a U-shape opening downward (refer to FIG. 8).

As illustrated in FIG. 8 where the filter 6 is disposed in the middle cavity portion 13 and the lid 4 is attached to the case 3, the upper filter 61 is located below the air inlet 51, the front filter 62 is located more to the rear than the air inlet 52, and the rear filter 63 is located more to the front than the air inlet 53.

As illustrated in FIG. 4 where the filter 6 is disposed in the middle cavity portion 13 and the lid 4 is removed from the case 3, the upper filter 61 is exposed from the opening 325. The length of the filter 6 in the front-rear direction is shorter than the length of the opening 325 in the front-rear direction. The length of the filter 6 in the left-right direction is shorter than the length of the opening 325 in the left-right direction. Therefore, the user can attach and detach the filter 6 to and from the middle cavity portion 13 via the opening 325.

As illustrated in FIG. 5, shape retainers 64 and 65 are provided at both left and right ends of the filter 6. The shape retainers 64 and 65 have a plate shape and are made of nonwoven fabric. The shape retainer 64 includes a shape retaining part 641 and a protrusion 642. The shape retainer 65 includes a shape retaining part 651 and a protrusion 652.

The shape retaining portions 641 and 651 have the same shape as the cross-sectional shape of the filter 6, and have a U-shape opening downward in the present embodiment. The shape retaining portion 641 is bonded to the left end of the filter 6. The shape retaining portion 651 is bonded to the right end of the filter 6. The shape retaining portions 641 and 651 retain the shape of the filter 6 so as to maintain the cross-sectional shape of the filter 6 in a U-shape. The protrusions 642 and 652 extend upward from the upper ends of the shape retaining portions 641 and 651, respectively. In a state where the filter 6 is disposed in the middle cavity portion 13 and the lid 4 is removed from the case 3, the protrusions 642 and 652 protrude upward relative to the upper surface of the upper filter 61. When the filter 6 is disposed in the middle cavity portion 13 and the lid 4 is attached to the case 3, the protrusion 642 is bent leftward and the protrusion 652 is bent rightward. The filter 6 is thus disposed in the middle cavity portion 13 with the protrusions 642 and 652 not protruding upward relative to the upper wall 32 (refer to FIG. 1).

Sealing members 66 and 67 are each bonded to one of left and right surfaces of the shape retaining portion 641. The sealing members 66 and 67 have the same shape as the cross-sectional shape of the filter 6, and in the present embodiment, have a U-shape opening downward. The seal members 66 and 67 are made of soft urethane foam. The sealing member 66 fills a gap between the shape retaining portion 641 and the partition wall 371 in the left-right direction to enhance airtightness in the housing 2. The seal member 67 fills a gap between the shape retaining portion 651 and the partition wall 372 in the left-right direction to enhance airtightness in the housing 2. The seal member 66 and 67 improves the air suction efficiency of the blower 81 and 82 through the filter 6.

Electrical Configuration of Air Purifier 1

As illustrated in FIG. 5, the air purifier 1 includes a slide switch 8 and a control board 9. The slide switch 8 is located at a lower right portion of the front wall 33, and is located below the middle wall 37 in the vertical direction. The user can switch the power supply of the air purifier 1 between ON and OFF by operating the slide switch 8 in the horizontal direction.

As illustrated in FIG. 6, the control board 9 is disposed in the accommodating portion 19. The control board 9 is provided with a control circuit (not shown), a USB connector 91, and a plurality of cable connectors 92. The USB connector 91 is exposed to the outside of the housing 2 through the recess 355. A USB cable (not shown) is connected to the USB connector 91 from the outside of the housing 2. Power is supplied to the air purifier 1 via the USB cable. Cables 93, 94, and 95 are each connected to a corresponding one of the cable connectors 92. The cables 93 and 94 illustrated in FIG. 5 connect the motors 812 and 822 to the control board 9, respectively. The cable 95 illustrated in FIG. 6 connects the slide switch 8 and the control board 9.

Method of Using Air Purifier 1

As illustrated in FIG. 1, the air purifier 1 is installed on the installation surface 99 such that the lower surface 21 of the housing 2 faces the installation surface 99. The installation surface 99 is a top plate of a desk, a floor, or other flat surface. The user connects a USB cable (not shown) to the USB connector 91. The air purifier 1 is thus brought into a state in which the air purifier 1 can be supplied with power. In this state, the user switches the power supply of the air purifier 1 from off to on by moving the slide switch 8 from left to right. As a result, power is supplied to the air purifier 1, and the air purifier 1 starts operating.

As illustrated in FIG. 6, the control board 9 drives the motors 812 and 822 to rotate the fans 813 and 823. The fan 813 rotates to generate a flow of air from the intake portion 816 toward the exhaust portion 817. The fan 823 rotates to generate a flow of air from the intake portion 826 toward the exhaust portion 827. Thus, air is sucked in into the middle cavity portion 13 from the outside of the housing 2 through the air inlets 51, 52, and 53.

The air is sucked in through the filter 6 into the cavity portion 13. Specifically, the air sucked in through the air inlet 51 into the middle cavity portion 13 passes through the upper filter 61 from the upper side to the lower side. The air sucked in through the air inlet 52 into the middle cavity portion 13 passes through the front filter 62 from the front side toward the rear side. The air sucked in through the air inlets 53 into the middle cavity portion 13 passes through the rear filter 63 from the rear side toward the front side. The filter 6 thus cleans the air sucked in into the cavity portion 13.

The air that has passed through the filter 6 flows toward the air outlet 55 or the air outlet 56. The air that has flowed toward the air outlet 55 flows from the intake portion 816 toward the exhaust portion 817. The air that has flowed toward the air outlet 56 flows from the intake portion 826 toward the exhaust portion 827. The air flowing from the intake portion 816 toward the exhaust portion 817 is blown out to the outside of the housing 2 through the air outlet 55 while being guided downward by the louver 71. The air that has flowed from the intake portion 826 toward the exhaust portion 827 is blown out to the outside of the housing 2 through the air outlet 56 while being guided downward by the louver 72.

Method of Replacing Filter 6

As illustrated in FIG. 1, the user presses the second end 412 of the grip portion 41 to the right so as to approach the first end 411. As a result, the engagement between the locking piece 413 and the upper wall 32 is released. In this state, the user lifts the grip portion 41 upward. Thus, the lid 4 is removed from the case 3.

As illustrated in FIG. 4, the user takes out the filter 6 from the cavity portion 13 to the outside of the case 3 through the opening 325 by holding the protrusions 642 and 652. The user attaches a new filter 6 to the cavity portion 13 from the outside of the case 3 through the opening 325 by holding protrusions 642 and 652.

As illustrated in FIG. 1, the user attaches the lid 4 to the case 3 while pressing the second end 412 of the grip portion 41 to the right. The user moves his/her hand off the grip portion 41. As a result, the second end 412 of the grip portion 41 is elastically deformed leftward, so that the second end 412 is away from the first end 411 and the locking piece 413 engages with the upper wall 32. When the lid 4 is attached to the case 3, the filter 6 is disposed in the middle cavity portion 13 in a state in which the protrusion 642 is bent leftward and the protrusion 652 is bent rightward. The user affixes a sticker (not shown) to the affixture portion 28 illustrated in FIG. 3. The sticker is affixed to the affixture portion 28 so as to cover the grip portion 41. Thus, the replacement of the filter 6 is completed.

### Operational Effects of Present Embodiment

The effects of the present embodiment described below are merely examples. Hereinafter, a direction in which each of the air inlets 51 to 53 is open, that is, an up-down direction or a front-rear direction is referred to as a “suction direction”, and a direction in which each of the air outlets 55 and 56 is open, that is, a left-right direction is referred to as a “blowout direction”. The air velocity at the air outlet 55 or the air outlet 56 is simply referred to as an “air outlet velocity”, and the air velocity at the air inlets 51 to 53 is simply referred to as an “air inlet velocity”.

In the present embodiment, since the air purifier 1 includes the two blowers 81 and 82, both the air outlet velocity and the air inlet velocity can be increased as compared with a case where one of the blowers 81 and 82 is omitted. Since the suction direction is orthogonal to the blowout direction, the air is blown out from the air outlets 55 and 56 so as to move away from the air inlets 51 to 53. Further, since the opening area of each of the air outlets 55 and 56 is smaller than the opening area of the air inlets 51, 52, and 53, the air outlet velocity is higher than the air inlet velocity. Therefore, the air blown out from each of the air outlets 55 and 56 moves further away from the air inlets 51 to 53 as compared with the case where the blow-out speed is lower than the suction speed. The air purifier 1 can thus reduce the likelihood that the air blown out from each of the air outlets 55 and 56 is sucked in again from the air inlets 51 to 53.

Two blowers 81 and 82 face each other in the left-right direction. Therefore, air flows in blowing directions along the left-right direction by the rotation of the fans 813 and 823. Thus, the air purifier 1 can increase the air outlet velocity as compared with a case where the two blowers 81 and 82 do not face each other in the left-right direction.

The air inlet 51 opens upward. The louvers 71 and 72 guide the air, which is blown out from the air outlets 55 and 56 by the two blowers 81 and 82, downward to the outside of the housing 2. Therefore, the air is blown out from each of the air outlets 55 and 56, which are apart from the air inlet 51, to the outside of the housing 2. The air purifier 1 can thus reduce the likelihood that the air blown out from each of the air outlets 55 and 56 is sucked in again from the air inlets 51 to 53.

For example, when the angle θ1 of the slats 713 to the left relative to the reference line V is smaller than 45 degrees, the amount of air, blown out through the air outlet 55, hitting the slats 713 may increase more than when the angle θ1 is equal to or greater than 45 degrees. As a result, there is a possibility that the air velocity at the air outlet 55 decreases. For example, when the angle θ1 of the slats 713 to the left relative to the reference line V is greater than 60 degrees, the louver 71 is less likely to downwardly guide the air blown out from the air outlet 55 than when the angle θ1 is 60 degrees or less. When the angle θ1 of the slat 713 to the left relative to the reference line V is 45 degrees or more and 60 degrees or less, the air purifier 1 can reduce the likelihood that the air blown from the air outlet 55 is sucked in again from the air inlet 51 while preventing a reduction in the air velocity at the air outlet 55. In this embodiment, the slats 713 and 723 are inclined 55 degrees relative to the suction direction. The air purifier 1 can thus reduce the likelihood that the air blown from each of the air outlets 55 and 56 is sucked in again from the air inlets 51 to 53 while preventing a reduction in the air outlet velocity. To enable the louver 71 to guide the air blown from the air outlet 55 downward, the angle θ1 of the slats 713 to the left relative to the reference line V may be greater than 55 degrees or smaller than 55 degrees. For the above-described reason, the angle θ1 of the slats 713 to the left relative to the reference line V is preferably 45 degrees or more and 60 degrees or less. As the angle θ1 of the slats 713 to the left relative to the reference line V is smaller, it is more difficult to visually recognize the inside of the housing 2 through the openings 715 in the left side view. Therefore, the smaller the angle θ1 of the slat 713 to the left relative to the reference line V is, the more the design of the air purifier 1 is improved. Further, in the air purifier 1, the smaller the angle θ1 of the slats 713 to the left relative to the reference line V is, the more easily a foreign substance such as a stick or dust can be prevented from entering the inside of the housing 2 through the openings 715. In contrast, the greater the angle θ1 of the slats 713 to the left relative to the reference line V is, the more the air purifier 1 can increase the air outlet velocity. The louver 72 can be modified in the same manner as the louver 71.

The first elastic members 716 and 717 are located between and in contact with the louver 71 and the blower 81. The first elastic members 726 and 727 are located between and in contact with the louver 72 and the blower 82. An impact may be applied to the louvers 71 and 72 due to, for example, a fall of the housing 2. In this case, the impact on the louvers 71 and 72 is absorbed by the first elastic members 716, 717, 726, and 727. This reduces the likelihood that the impact on the louvers 71 and 72 is directly transmitted to the blowers 81 and 82. Therefore, the air purifier 1 can reduce failures of the blowers 81 and 82 due to the impact on the louvers 71 and 72.

The protrusions 381 and 382 protrude toward the fans 813 and 823, respectively. The distal ends 383 and 384 of the protrusions 381 and 382 are each located at a position spaced from the fans 813 and 823. For example, when the housing 2 falls, the fans 813 and 823 may move from the support portions 811 and 821 toward the partition walls 371 and 372 so as to remove from the output shafts of the motors 812 and 822, respectively. In this case, the fans 813 and 823 come into contact with the protrusions 381 and 382, respectively. This prevents the fans 813 and 823 from being detached from the support portions 811 and 821. The distal ends 383 and 384 of the protrusions 381 and 382 are each located at a position spaced from a corresponding one of the fan 813 and 823, thus preventing the protrusions 381 and 382 from interfering with the rotation of the fans 813 and 823. The air purifier 1 can thus use the protrusions 381 and 382 to prevent the fans 813 and 823 from being detached from the support portions 811 and 821, while preventing the rotation of the fans 813 and 823 from being hindered by the protrusions 381 and 382.

The contact portions 391 and 392 come into contact with the support portions 811 and 821, respectively. The contact portions 391 and 392 thus prevent the support portions 811 and 821 from moving relative to the housing 2. Since the length H1 from the partition wall 371 to the distal end 383 of the protrusion 381 is shorter than the length H2 from the partition wall 371 to the distal end 393 of the contact portion 391, the contact portion 391 comes into contact with the support portion 811 before the protrusion 381 comes into contact with the fan 813. Since the length H3 from the partition wall 372 to the distal end 384 of the protrusion 382 is shorter than the length H4 from the partition wall 372 to the distal end 394 of the contact portion 392, the contact portion 392 comes into contact with the support portion 821 before the protrusion 382 comes into contact with the fan 823. The air purifier 1 can thus prevent the rotation of the fans 813 and 823 from being hindered by the protrusions 381 and 382, while using the contact portions 391 and 392 to prevent the support portions 811 and 821 from moving relative to the housing 2.

The second elastic members 215 to 218 are located on the lower surface 21 of the housing 2. The air purifier 1 may be installed on the installation surface 99 such that the lower surface 21 of the housing 2 and the installation surface 99 face each other. In this case, the second elastic members 215 to 218 are in contact with the installation surface 99, thus preventing the air purifier 1 from slipping relative to the installation surface 99 due to friction between the second elastic members 215 to 218 and the installation surface 99. Therefore, the air purifier 1 can improve the tackiness.

The coefficient of static friction of the second elastic members 215 to 218 is greater than the coefficient of static friction of the lower surface 21 of the housing 2. Thus, the friction between the installation surface 99 and the second elastic members 215 to 218 is high as compared to a case where the coefficient of static friction of the second elastic members 215 to 218 is smaller than the coefficient of static friction of the lower surface 21 of the housing 2, the air purifier 1 is further prevented from slipping relative to the installation surface 99. Therefore, the air purifier 1 can further improve the tackiness.

The second elastic members 215 to 218 have a 25 percent compression-deflection value of 0.133 N/mm2 or less per unit area. Vibration during the operation of the air purifier 1 is transmitted to the installation surface 99 via the second elastic members 215 to 218. When the 25 percent compression-deflection value of the second elastic members 215 to 218 is 0.133 N/mm2, the air purifier 1 can control the vibration transmissibility at the installation surface 99, to which vibration is transmitted via the second elastic members 215 to 218, to less than 100%. Therefore, the air purifier 1 can prevent the vibration during the operation from being transmitted to the installation surface 99. The vibration transmissibility of the second elastic members 215 to 218 is the ratio of the magnitude of the vibration transmitted from the second elastic members 215 to 218 to the installation surface 99 to the magnitude of the vibration transmitted from the housing 2 to the second elastic members 215 to 218.

The lid 4 has the air inlet 51 and the grip portion 41. Therefore, the user can remove the lid 4 from the case 3 by holding the grip portion 41. Therefore, the air purifier 1 can prevent the user from touching the air inlet 51 when the lid 4 is removed from the case 3. Therefore, the air purifier 1 can reduce the likelihood that the air inlet 51 is fouled when the lid 4 is attached to and detached from the case 3 by the user. The air purifier 1 can reduce the likelihood that the user directly touches the air inlet 51 through which the air before purification passes, and is hygienic.

The air inlet 325 is open in the up-down direction. Therefore, when the air purifier 1 is installed on the installation surface 99 such that the lower surface 21 of the housing 2 and the installation surface 99 face each other, the lid 4 is located on a side of the case 3 opposite to the installation surface 99. Therefore, in the air purifier 1, the lid 4 can be easily removed from the case 3.

The housing 2 has the affixture portion 28. Therefore, when the sticker is affixed to the affixture portion 28, the grip portion 41 is covered with the sticker. Therefore, the air purifier 1 can prevent the grip portion 41 from being unintentionally touched by the user.

### Correspondence Description

In the above embodiment, the air inlets 51 to 53 each correspond to an “inlet” of the present disclosure. The air outlets 55 and 56 each correspond to an “outlet” of the present disclosure. The housing 2 corresponds to a “housing” of the present disclosure. The cavity portion 10 corresponds to a “cavity portion” of the present disclosure. The blowers 81 and 82 correspond to “blowers” of the present disclosure. The fans 813 and 823 each correspond to a “fan” of the present disclosure. The filter 6 corresponds to a “filter” of the present disclosure.

The louvers 71 and 72 each correspond to a “louver” of the present disclosure. The first elastic members 716, 717, 726, 727 each correspond to a “first elastic member” of the present disclosure. The support portions 811 and 821 each correspond to a “support portion” of the present disclosure. The left cavity portion 11 and the right cavity portion 12 each correspond to a “first cavity portion” of the present disclosure. The middle cavity portion 13 corresponds to a “second cavity portion” of the present disclosure. The partition walls 371 and 372 each correspond to a “partition wall” of the present disclosure. The protrusions 381 and 382 each correspond to a “protrusion” of the present disclosure. The contact portions 391 and 392 each correspond to a “contact portion” of the present disclosure.

The second elastic members 215 to 218 each correspond to a “second elastic member” of the present disclosure. The lower surface 21 of the housing 2 corresponds to a “support surface portion” of the present disclosure. The opening 325 corresponds to an “opening” of the present disclosure. The case 3 corresponds to a “case” of the present disclosure. The lid 4 corresponds to a “lid” of the present disclosure. The grip portion 41 corresponds to a “grip portion” of the present disclosure. The affixture portion 28 corresponds to an “affixture portion” of the present disclosure.

### First Modification

An air purifier 1A according to a first modification will be described with reference to FIG. 9. The air purifier 1A is different from the air purifier 1 in that the air purifier 1A further includes blowers 83 and 84 in addition to the blowers 81 and 82, and a partition portion 375A, which is different from the partition portion 375 in shape. That is, the number of blowers is not limited to two, and may be three or more. Other structures of the air purifier 1A are identical to those of the air purifier 1. The filter 6 is not illustrated in FIG. 9 (the same applies to FIG. 10 described later).

The partition portion 375A has a middle partition wall 376A, a left partition wall 377A, and a right partition wall 378A. The middle partition wall 376A, the left partition wall 377A, and the right partition wall 378A have a plate shape and extend in the front-rear direction. The middle partition wall 376A extends upward from the center of the middle wall 37 in the left-right direction. The left partition wall 377A extends obliquely upward from the right of the partition wall 371 on the middle wall 37 toward the right of an upper end of the middle partition wall 376A. The right partition wall 378A extends obliquely upward to the left of the partition wall 372 on the middle wall 37 toward the left of an upper end of the middle partition wall 376A. The upper ends of the inner partition wall 376A, the left partition wall 377A, and the right partition wall 378A are each located at a position spaced downward from the upper wall 32.

The middle cavity portion 13 is divided into partitioned cavity portions 131, 132, 133, and 134 by the middle partition wall 376A, the left partition wall 377A, the right partition wall 378A, and the partition walls 371 and 372. The partitioned cavity portion 131 is a space defined between the partition wall 371 and the left partition wall 377A in the left-right direction in the middle cavity portion 13. The partitioned cavity portion 132 is a space defined between the left partition wall 377A and the middle partition wall 376A in the left-right direction in the middle cavity portion 13. The partitioned cavity portion 133 is a space defined between the middle partition wall 376A and the right partition wall 378A in the left-right direction in the middle cavity portion 13. The partitioned cavity portion 134 is a space defined between the right partition wall 378A and the partition wall 372 in the left-right direction in the middle cavity portion 13. The middle wall 37 has an opening 378 defined between lower ends of the middle partition wall 376A and the left partition wall 377A in the left-right direction. The partitioned cavity portion 132 communicates with the accommodating portion 19 via the opening 378. The middle wall 37 has an opening 379 defined between lower ends of the middle partition wall 376A and the right partition wall 378A in the left-right direction. The partitioned cavity portion 133 communicates with the accommodating portion 19 via the opening 379.

The lower surface 21 of the housing 2 has air outlets 57 and 58. That is, the number of air outlets is not limited to two, and may be three or more. The air purifier 1A may omit one of the air outlets 55 and 56.

The air outlet 57 is an opening and is defined in the lower wall 31 between the middle partition 376A and the left partition 377A in the left-right direction. The air outlet 58 is an opening and is defined in the lower wall 31 between the middle partition 376A and the right partition 378A in the left-right direction. The opening area of the air outlet 57 is smaller than the opening area of the air inlets 51, 52, and 53 illustrated in FIGS. 1 to 3. The opening area of the air outlet 58 is smaller than the opening area of the air inlets 51, 52, and 53 illustrated in FIGS. 1 to 3.

The air purifier 1A includes blowers 83 and 84. The blowers 83 and 84 are fixed to the accommodating portion 19. The blower 83 is located between the lower ends of the middle partition wall 376A and the left partition wall 377A in the left-right direction. An intake portion 831 of the blower 83 faces the opening 378 in the up-down direction. An exhaust portion 832 of the blower 83 faces the air outlet 57 in the vertical direction. The blower 84 is located between the lower ends of the middle partition wall 376A and the right partition wall 378A in the left-right direction. An intake portion 841 of the blower 84 faces the opening 379 in the up-down direction. An exhaust portion 842 of the blower 84 faces the air outlet 58 in the vertical direction.

Hereinafter, portions of the air inlet 51, the air inlet 52 illustrated in FIG. 1, and the air inlet 53 illustrated in FIG. 2, which are connected to the partitioned cavity portions 131, 132, 133, and 134, are referred to as a “first partial inlet”, a “second partial inlet”, a “third partial inlet”, and a “fourth partial inlet”, respectively.

According to the structure of the first modification, the blower 81 sucks in air into the partitioned cavity portion 131 through the first partial inlet of the air inlets 51 to 53. The air passes through the filter 6 and is purified in the process of being sucked in into the partitioned cavity portion 131. The blower 81 blows the air purified in the partitioned cavity portion 131 leftward from the air outlet 55.

The blower 82 sucks in air into the partitioned cavity portion 134 through the fourth partial inlet of the air inlets 51 to 53. The air passes through the filter 6 and is purified in the process of being sucked in into the partitioned cavity portion 134. The blower 82 blows the air purified in the partitioned cavity portion 134 rightward from the air outlet 56.

The blower 83 sucks in air into the partitioned cavity portion 132 through the second partial inlet of the air inlets 51 to 53. The air passes through the filter 6 and is purified in the process of being sucked in into the partitioned cavity portion 132. The blower 83 blows the air purified in the partitioned cavity portion 132 downward through the opening 378 from the air outlet 57.

The blower 84 sucks in air into the partitioned cavity portion 133 through the third partial inlet of the air inlets 51 to 53. The air passes through the filter 6 and is purified in the process of being sucked in into the partitioned cavity portion 133. The blower 84 blows the air purified in the partitioned cavity portion 133 downward through the opening 379 from the air outlet 58.

As described above, since the air purifier 1A has the four blowers 81 to 84, the amount of air sucked in from the air inlets 51 to 53 per unit time can be increased as compared with the air purifier 1. The air purifier 1A causes the airflows formed by driving each of the blowers 81 to 84 to flow through the partitioned cavity portions 131 to 134. For this reason, a mutual influence on the airflows in the middle cavity portion 13 in response to the driving of the blowers 81 to 84 is reduced by the partition portion 375A. The air purifier 1A can thus cause air to flow smoothly in the middle cavity portion 13. Therefore, the air purifier 1A can purify air more efficiently.

Further, since the opening area of each of the air outlets 55, 56, 57, and 58 is smaller than the opening area of the air inlets 51, 52, and 53, the air outlet velocity is higher than the air inlet velocity. The air purifier 1A can thus reduce the likelihood that the air blown from each of the air outlets 55, 56, 57, and 58 is sucked in again from the air inlets 51 to 53, similarly to the air purifier 1.

In the first modification, the upper ends of the middle partition wall 376A, the left partition wall 377A, and the right partition wall 378A may be connected to each other. The middle partition wall 376A, the left partition wall 377A, and the right partition wall 378A may extend such that their upper ends are to be located in contact with the lid 4.

### Second Modification

An air purifier 1B according to a second modification will be described with reference to FIG. 10. The air purifier 1B is different from the air purifier 1 in that the housing 2 accommodates ducts 38 and 39. The ducts 38 and 39 are tubular and each have two openings.

One opening of the duct 38 is connected to the left end 323 of the upper wall 32. The duct 38 is bent, extending from top to bottom in a direction from right to left. The space inside the duct 38 is the left cavity portion 11. The other opening of the duct 38 is the air outlet 55 and is located above the lower surface 21 of the housing 2. The exhaust portion 817 of the blower 81 faces the air outlet 55 in the up-down direction.

One opening of the duct 39 is connected to the right end 324 of the upper wall 32. The duct 39 is bent, extending from top to bottom in a direction from left to right. The space in the duct 39 is the right cavity portion 12. The other opening of the duct 38 is the air outlet 56 and is located above the lower surface 21 of the housing 2. The exhaust portion 827 of the blower 82 faces the air outlet 56 in the up-down direction.

According to the structure of the second modification, the blowers 81 and 82 each suck in air into the middle cavity portion 13 through the air inlets 51 to 53. The air passes through the filter 6 and is purified in the process of being sucked in into the partitioned cavity portion 13. The blowers 81 and 82 blow the air purified in the middle cavity portion 13 downward from the air outlets 55 and 56, respectively. In this case, the louvers 71 and 72 may be omitted from the air purifier 1B.

Since the air purifier 1B is configured such that the opening area of each of the air outlets 55 and 56 is smaller than the opening area of the air inlets 51, 52, and 53, the air outlet velocity is higher than the air inlet velocity. The air purifier 1B can thus reduce the likelihood that the air blown from each of the air outlets 55 and 56 is sucked in again from the air inlets 51 to 53, as with the air purifier 1.

### Other Modifications

The present disclosure can be variously modified from the above embodiment in addition to the first modification and the second modification. The above-described embodiment, the first modification, the second modification, and the modifications described below can be appropriately combined with each other as long as no contradiction occurs. The air purifier 1 may include a button switch, a touch screen, and an infrared switch, instead of or in addition to the slide switch 8. In this case, the coefficient of static friction of the second elastic members 215 to 218 is of such a magnitude that the air purifier 1 does not move relative to the installation surface 99 in response to a switch operation performed by the user. In this case, the user can operate the switch without supporting the housing 2.

The air purifier 1 may be installed such that any of the lower surface 21, the upper surface 22, the front surface 23, the rear surface 24, the left surface 25, and the right surface 26 of the housing 2 faces the installation surface 99. The air purifier 1 may be used without being installed on the installation surface 99. For example, the air purifier 1 may be used in a state where the lower surface 21 of the housing 2 is fixed to a microphone stand such that a longitudinal surface of the housing 2 is parallel to the microphone stand. The outer shape of the housing 2 is not limited to a rectangular parallelepiped shape, and may be a spherical shape, a cylindrical shape, or a prismatic shape (such as a triangular prism). The directions in which the air inlets 51, 52, and 53 are open may not be orthogonal to, but should at least cross, the directions in which the air outlets 55 and 56 are open.

One or two of the air inlets 51, 52, and 53 may be omitted from the housing 2. For example, only the air inlets 52 and 53 of the air inlets 51, 52, and 53 may be defined in the housing 2, and the air inlet 51 may be omitted. For example, only the air inlet 51 of the air inlets 51, 52, and 53 may be defined in the housing 2, and the air inlets 52 and 53 may be omitted. An air inlet may be defined in the lower surface 21 in addition to the upper surface 22, the front surface 23, and the rear surface 24 of the housing 2. The air inlet may be located across a boundary line between adjacent walls, for example, the upper wall 32 and the front wall 33.

The rotation shafts of the fans 813 and 823 may extend obliquely upward or downward relative to the left-right direction, or may extend obliquely forward or rearward relative to the left-right direction. The blowers 81 and 82 may be provided rotatably relative to the housing 2. In this case, the user may adjust the direction of the airflow generated by the rotation of the fans 813 and 823 by rotating the blowers 81 and 82. The blowers 81 and 82 are not limited to an axial flow type, and may be a mixed flow type, a centrifugal type, or other flow type.

In the above embodiment, the opening 325 is defined in the upper wall 32. On the other hand, the opening 325 may be defined in one or more of the lower wall 31, the front wall 33, the rear wall 34, the left wall 35, and the right wall 36 instead of or in addition to the upper wall 32, or may be located across boundary line between adjacent walls. When a plurality of openings 325 are formed, a plurality of lids 4 may be provided so as to be each attachable to and detachable from a corresponding opening 325 of the plurality of openings 325.

The cross-sectional shape of the filter 6 is not limited to a U-shape, and may be any shape such as a circular shape, an elliptical shape, or a V-shape. The upper filter 61, the front filter 62, and the rear filter 63 may be configured separately from each other. The filter 6 may not be located in the middle cavity portion 13, and may be located, for example, in each of the left cavity portion 11 and the right cavity portion 12. In this case, for example, one filter 6 may be located between the blower 81 and the air outlet 55 and the other filter 6 may be located between the blower 82 and the air outlet 56 in the left-right direction. That is, the filter 6 may not be located between the blowers 81 and 82 in the left-right direction. The filter 6 is not limited to the HEPA filter, and may be an ULPA filter or other type of filter.

Instead of the second elastic members 215 to 218, adhesive members may be fixed to the recesses 211 to 214. In this case, the air purifier 1 can improve the tackiness. The air purifier 1 may omit some or all of the second elastic members 215 to 218. The number of second elastic members may be five or more. The positions where the second elastic members 215 to 218 are provided are not limited to those in the above embodiment. For example, the second elastic member may be fixed to the entire lower surface 21 of the housing 2. The second elastic member may be located at the same relative position as the middle cavity portion 13 in the left-right direction. Specifically, the second elastic member on the lower surface 21 of the housing 2 may be located at the same relative position as the partition portion 375 in the left-right direction, may be located at the same relative position as the partition portion 375 in the front-rear direction, or may be located at a position overlapping the partition portion 375 in a plan view. The partition portion 375 may be vibrated by the air sucked in from the air inlet 51. Since the second elastic member is located below the partition portion 375, the air purifier 1 easily absorbs the vibration of the partition portion 375 by the second elastic member. The recesses 211 to 214 may be omitted from the lower surface 21, and the second elastic member may be directly fixed to at least a part of the lower surface 21. In this case, the lower surface 21 corresponds to a “support surface” of the present disclosure.

The second elastic members 215 to 218 may be rubber such as non-foamed urethane, foamed silicon, or non-foamed silicon, or may be a sponge, a spring, felt, or another elastic member. The second elastic members 215 to 218 may have a 25 percent compression-deflection value of 0.133 N/mm2 or more per unit area.

The coefficient of static friction of each of the second elastic members 215, 216, 217, and 218 may be equal to or less than the coefficient of static friction of the lower surface 21 of the housing 2. The coefficient of static friction of each of the second elastic members 215, 216, 217, and 218 may be equal to or less than the coefficient of static friction of the bottom surface of the recess 211.

The number of the first elastic members may be three or more. The air purifier 1 may omit some or all of the first elastic members 716 and 717. In this case, the louver 71 may be in direct contact with the blower 81. The louver 71 and the blower 81 may be fixed to the case 3 so as to be spaced from each other in the left-right direction. The first elastic members 716 and 717 may be fixed to any position on the right surface of the louver 71. The first elastic members 716 and 717 may be fixed to the right end 814 of the support portion 811 in the blower 81 without being fixed to the right surface of the louver 71, or may be fixed to both the right surface of the louver 71 and the right end 814 of the support portion 811. The first elastic members 726 and 727 may be modified in the same manner as the first elastic members 716 and 717. The first elastic members 716, 717, 726, and 727 are not limited to a sponge, and may be rubber, a spring, felt, or another elastic member.

The method of fixing the blower 81 to the case 3 is not limited to the above-described embodiment. For example, the air purifier 1 may omit some or all of the contact portions 391, or may include five or more contact portions 391. The contact portions 391 may protrude upward from the middle wall 37. In this case, the position of the blower 81 in the left-right direction is determined by the support portion 811 coming into contact with the contact portions 391 from the left side. The fixing method of the blower 82 to the case 3 can be changed in the same manner as the blower 81.

The grip portion 41 is not limited to a snap fit. That is, in the above-described embodiment, the grip portion 41 has a function of being gripped by the user and a function of maintaining a state where the lid 4 is attached to the case 3. In contrast, for example, the grip portion 41 may extend upward from a position different from the air inlet 51 in the lid 4 and may not have a function of maintaining a state of being attached to the case 3.

In the above embodiment, the lid 4 is completely removable from the case 3. Alternatively, the lid 4 may be connected to the case 3 via, for example, a hinge. “The lid 4 is detachable from the case 3” means both a case where the lid 4 is attachable to the case 3 and completely detachable from the case 3, and a case where the lid 4 is openable and closable with respect to the case 3 in a state where a part of the lid 4 is connected to the case 3.

The housing 2 should have at least one air outlet having an opening area smaller than the opening area of the air inlets 51, 52, and 53. That is, one of the air outlets 55 and 56 should have an opening area smaller than the opening area of the air inlets 51 to 53.

The louver may include a slat inclined 45 degrees to 60 degrees relative to the suction direction.

While the disclosure has been described in detail with reference to the specific embodiment thereof, this is merely an example, and various changes, arrangements and modifications may be applied therein without departing from the spirit and scope of the disclosure.

What is claimed is:

1. An air purifier comprising:
a housing having an air inlet, an air outlet, and a cavity portion connecting the air inlet and the air outlet;
two blowers disposed in the cavity portion and each including a fan rotatable to generate a flow of air; and
a filter disposed in the cavity portion,
wherein the two blowers are each configured to, during rotation of the fan, suck in air from outside of the housing through the air inlet into the cavity portion, and blow the air having passed through the filter from the air outlet to the outside of the housing,
wherein a direction in which the air inlet is open crosses a direction in which the air outlet is open, and
wherein an opening area of the air outlet is smaller than an opening area of the air inlet.

2. The air purifier according to claim 1, wherein the two blowers face each other in the direction in which the air outlet is open.

3. The air purifier according to claim 1, further comprising a louver engaged in the air outlet and configured to guide the air blown from the air outlet in a direction away from the housing.

4. The air purifier according to claim 1, further comprising:
a louver engaged in the air outlet; and
a first elastic member located between and in contact with the louver and one of the two blowers.

5. The air purifier according to claim 3, further comprising a first elastic member located between and in contact with the louver and one of the two blowers.

6. The air purifier according to claim 1, wherein
the two blowers each include a support portion supporting the fan,
the housing includes a partition wall dividing the cavity portion into a first cavity portion and a second cavity portion,
one of the two blowers is disposed in the first cavity portion,
the filter is disposed in the second cavity portion,
the partition wall has a protrusion protruding toward the fan, and
the protrusion has a distal end spaced from the fan.

7. The air purifier according to claim 6, wherein
the partition wall includes a contact portion protruding toward the supporting portion, the contact portion having a distal end in contact with the supporting portion, and
a length from the partition wall to the distal end of the protrusion is shorter than a length from the partition wall to the distal end of the contact portion.

8. The air purifier according to claim 1, further comprising a second elastic member, wherein the housing has a support surface on which the second elastic member is located.

9. The air purifier according to claim 8, wherein the second elastic member has a static friction coefficient greater than a static friction coefficient of the support surface.

10. The air purifier according to claim 8, wherein the second elastic member has a 25 percent compression-deflection value of 0.133 N/mm2 or less per unit area.

11. The air purifier according to claim 1, wherein
the housing includes a case having an opening for attaching and detaching the filter, and a cover attachable to the case to cover the opening, and
the cover has the air inlet and includes a grip portion for removing the cover from the case.

12. The air purifier according to claim 11, wherein
the housing has a support surface on which the second elastic member is located, and
the opening is defined in a surface of the housing opposite to the support surface.

13. The air purifier according to claim 11, wherein the housing has an affixture portion for affixing a sticker to cover the grip portion.

14. The air purifier according to claim 1, further comprising a further blower,
wherein the housing has a first surface and a second surface opposite to the first surface, the first surface having the air inlet, the second surface having a second air outlet,
wherein the further blower includes an exhaust portion facing the second air outlet, and
wherein an opening area of the second air outlet is smaller than the opening area of the air inlet.

15. The air purifier according to claim 14, wherein
the housing has a partition wall dividing the cavity portion into a first cavity portion and a second cavity portion,
one of the two blowers is configured to suck in the air through the air inlet and the filter into the first cavity portion, and
the further blower is configured to suck in the air through the air inlet and the filter into the second cavity portion.

16. The air purifier according to claim 1, wherein
the housing has a duct that is tubular and bent,
the duct has an inside space that is the cavity portion,
the duct has an opening located above a lower surface of the housing,
the opening of the duct is the air outlet, and
the two blowers each include an exhaust portion facing the air outlet and each configured to blow the air from the air outlet to the outside of the housing downward.
